(19) United States
(12) Patent Application Publication
(10) Pub. No.: US 2020/0305736 A1
(43) Pub. Date: Oct. 1, 2020

(54) METHODS, SYSTEMS, AND COMPUTER READABLE MEDIA FOR MEASURING SYSTEMIC VASCULAR RESISTANCE

(71) Applicant: The Trustees of the University of Pennsylvania, Philadelphia, PA (US)

(72) Inventors: Jennifer Beth Monti, Philadelphia (US); Sarah Mary Nims, Philadelphia (US); Kelly Michelle Rogers, Lansdale (US); Emily Margaret Olson, Cincinnati (US); Veena Trish Krish, Philadelphia (US)

(21) Appl. No.: 16/089,930

(22) PCT Filed: Mar. 31, 2017
(86) PCT No.: PCT/US2017/025402

§ 371 Date: Sep. 28, 2018

Related U.S. Application Data
(60) Provisional application No. 62/316,889, filed on Apr. 1, 2016.

(51) Int. Cl. A61B 5/024; A61B 5/00; A61B 5/02; A61B 5/021; A61B 5/1455

(52) U.S. Cl. CPC A61B 5/02416; A61B 5/742; A61B 5/02007; A61B 5/1455; A61B 5/02116; A61B 5/02125; A61B 5/7235

(57) ABSTRACT

The subject matter described herein relates to methods, systems, and computer readable media lor measuring systemic vascular resistance (SVR). In some examples, a method for measuring SVR includes determining, by a computer system coupled to a photoplethysmography (PPG) sensor and a display device, a plurality of wave parameters from a cardiac waveform signal detected by the PPG sensor, wherein the wave parameters include at least a systolic peak amplitude, a diastolic peak amplitude, and a dicrotic notch amplitude. The method includes determining, by the computer system, an SVR value based on the wave parameters. The method includes displaying the SVR value on the display device or other available screen.

### PRIORITY CLAIM

This application claims the benefit of U.S. patent application Ser. No. 62/316,889, filed Apr. 1, 2016, the disclosure of which is incorporated by reference in its entirety.

### TECHNICAL FIELD

The subject matter described herein relates generally to measuring systemic vascular resistance (SVR). More particularly, the subject matter described herein relates to methods, systems, and computer readable media for a clinical, noninvasive assessment of SVR.

### BACKGROUND

Systemic vascular resistance (SVR) is a measure of vascular tone that varies depending on the overall status of the patient. High SVR can be life-threatening, and low SVR can be life-threatening.

Accordingly, diagnosing conditions efficiently by quickly screening for possible causes of the patient's condition is vital. The measurement of systemic vascular resistance (SVR) has the potential to be a powerful tool for screening for sepsis and heart failure, conditions marked by abnormally low and high SVR values, respectively. Currently, these conditions lack an efficient screening tool. SVR is a measure of cardiovascular function and overall vascular tone, and it is calculated by:

$$SVR = 80 \cdot \frac{MAP - CVP}{CO} ,$$

where MAP is mean arterial pressure, CVP is central venous pressure, and CO is cardiac output. A person of good cardiovascular health will have an SVR between 900^{dyne·s}/cm⁵ and 1200^{dyne·s}/cm⁶.

An abnormally low SVR is an indicator of sepsis, which is a life-threatening complication of an infection. A sepsis patient experiences a release of vasodilating chemicals, causing a drop in MAP and consequently a drop in SVR, as per the formula above. A patient can receive the diagnosis of sepsis if they present with any two of the following symptoms: a body temperature above 38.3° C. or below 36° C., a heart rate higher than 90 beats per minute, a respiratory rate higher than 20 breaths per minute, and probable or confirmed infection. The ambiguous nature of these symptoms leads to frequent missed or delayed diagnoses of the condition. Early detection and treatment of sepsis is crucial for a positive outcome, so it is likely that the current 15% mortality rate for 260,000 sepsis cases seen annually in U.S. emergency departments could be reduced if it were diagnosed earlier.

Conversely, abnormally high SVR is an indicator of congestive heart failure (CHF), which is a prevalent condition in the U.S., affecting 5.1 million people and expected to grow 46% by 2030.

Congestive heart failure (CHF) is a huge worldwide unsolved health burden: CHF is a disease that results in recurrent decompensation and inpatient medical treatment. CHF is a leading reason for hospital admission and thirty day re-admission, and is one of the most expensive conditions to treat in the United States.'

Current practices of CHF diagnosis rely on signs and symptoms that are vague and non-specific. This results in delayed diagnosis, inappropriate hospital discharge, and a dismal readmission rate of over 50% within six months of a heart failure diagnosis.² ADHF hospitalization is a predictor for death.³ It has been found that the 60-day mortality in patients after ADHF hospitalization is between 8% and 20% depending on the population studied.⁴

An increase in SVR is a hemodynamic hallmark of CHF exacerbation, but there is no simple way to measure this in the clinic or hospital. Cardiovascular physiology requires the body to seek a consistent mean arterial pressure (MAP), which is directly proportional to stroke volume, heart rate and SVR. Patients with heart failure cannot augment their stroke volume, and in an effort to maintain MAP, increase the heart rate and SVR. This leads to a vicious cycle; an increase in the resistance in the vessels that take blood away from the heart will cause the heart to ‘back up’ fluid into the lungs, leading to decompensation. Currently, the only method to measure SVR is through a right heart catheterization (RHC). However, this is expensive and invasive, and therefore reserved for only a fraction of CHF patients that are already known to be decompensated. There is no widely available non-invasive device for estimating SVR.

Furthermore, SVR is calculated from three measure variables (mean pulmonary artery pressure, pulmonary capillary wedge pressure, and cardiac output), each with its own error. There is no way to directly measure SVR. Given that SVR estimates from RHC can vary greatly depending on the positioning of the patient, time of day of the study, presence of afterload modifying medications, volume status, and phase of the respiratory cycle. patient temperature, and hematocrit⁵, a non-invasive device that gives an estimate within range of the RHC derived SVR would be useful. Discussions with clinicians indicate that RHC estimates of SVR vary up to 20% depending on the factors noted above.

Knowing the SVR of a patient would enable physicians to rule out or further investigate possibilities of CHF or sepsis, and to routinely estimaste SVR throughout a hospitalization. Currently, the SVR measurement cannot be utilized as an screening tool due to the time, cost, and risk-to-patient associated with the current means of measuring SVR, which is a right heart catheterization (RHC). This invasive procedure costs about $2000, and like other invasive procedures, has risks of infection or complication.

Photoplethysmography (PPG) technology can provide noninvasive hemodynamic information. The PPG sensor is typically placed on the finger pad where a source emits red, green or IR light (λ=1-10³ μm) (or light at any appropriate wavelength, e.g., red, green, IR, or combinations of these) and a detector senses the amount of the incident light reflected. Because blood absorbs light at these wavelengths, the change in amount of light reflected correlates to changes in blood volume. The output of the sensor is a pulse wave whose characteristics are affected by cardiovascular and arteriolar properties.

Today, the only widely-used clinical application of PPG is in monitoring blood oxygen saturation level, or SpO2, via a pulse oximeter. Although many researchers have established relationships between SVR value and PPG features, such as the relative position and/or amplitudes of the key points of the wave depicted in FIG. 1, no point-of-care or clinical device employing this relationship to report SVR has been developed. Lee et al. has developed a classifier that uses multiple features and can identify low SVR with a specificity of 86% and sensitivity of 85% and high SVR with a specificity of 93% but sensitivity less than 60%, and other groups have published work in this area as well.

The only current device that provides a non-invasive measure of SVR comes from a Netherlands-based company and is used in the U.S. solely for research purposes. This device, Finometer Pro, utilizes both a finger pressure cuff and PPG to report several hemodynamic metrics including SVR and CO, but its primary purpose is to reliably measure blood pressure. It calculates SVR as the ratio of MAP to CO, assuming zero central venous pressure. However, the Finometer Pro's measure of CO has a 20% error, and this error propagates to the SVR calculation. Additionally, the assumption of zero venous pressure is often invalid, especially for heart failure patients. The Finometer Pro also weighs approximately 35 pounds and costs over $10,000.

Noting these shortcomings, there is a clinical need for a point-of-care screening tool to estimate SVR by non-invasive methods.

### SUMMARY

This document describes a device that calculates SVR that is point-of-care, affordable, and acceptable for clinical application. The device can be used easily as a screening tool in all care settings. The device can be configured, by virtue of appropriate programming and sensor selection, so that its accuracy is acceptable for clinical use.

The subject matter described herein relates to methods, systems, and computer readable media for measuring systemic vascular resistance (SVR). In some examples, a method for measuring SVR includes determining, by a computer system coupled to a photoplethysmography (PPG) sensor and a display device, a plurality of wave parameters from a cardiac waveform signal detected by the PPG sensor, wherein the wave parameters include at least a systolic peak amplitude, a diastolic peak amplitude, and a dicrotic notch amplitude. The method includes determining, by the computer system, an SVR value based on the wave parameters. The method includes displaying the SVR value on the display device.

The subject matter described in this specification includes both self-contained point of care devices and other devices and systems for determining SVR values. In some examples, a system includes a device having a PPG sensor and a computer system (e.g., a tablet or laptop computer) for receiving measurements from the PPG sensor. The computer system is programmed to determine SVR values based on wave parameters determined from the measurements from the PPG sensor and to display an indicator based on the SVR value. In some examples, the computer system or the device having the sensor transmits the measurements from the PPG sensor to a cloud computing server that determines the SVR value. For example, the cloud computing server can then send the SVR value back to the computer system for display or send an indicator based on the SVR value back to the computer system for display, or the cloud computing server can store the SVR value for later use. The SVR values may be used, e.g., by a health professional, as one of the earliest physiologic changes in one or more acute and/or chronic illnesses. Examples of illnesses where SVR values may be useful for diagnosis include congestive heart failure, sepsis, kidney disease, and liver disease.

The subject matter described in this specification may be implemented in hardware, software, firmware, or combinations of hardware, software and/or firmware. In some examples, the subject matter described in this specification may be implemented using a non-transitory computer readable medium storing computer executable instructions that when executed by one or more processors of a computer cause the computer to perform operations. Computer readable media suitable for implementing the subject matter described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application specific integrated circuits. In addition, a computer readable medium that implements the subject matter described in this specification may be located on a single device or computing platform or may be distributed across multiple devices or computing platforms.

### BRIEF DESCRIPTION OF DRAWINGS

FIG. 1 is an example PPG waveform;

FIG. 2 is a block diagram illustrating the use of an example device for measuring SVR values;

FIGS. 3A-C illustrate aspects of the process for developing a model for determining SVR values from cardiac waveforms and for implementing devices to use the model;

FIG. 4 shows an electrical schematic of an example implementation of the EasySVR device;

FIG. 5A depicts the inside of an example EasySVR;

FIG. 5B depicts the outside of the example EasySVR;

FIG. 6 is a flowchart showing the use of the device and user interface;

FIG. 7 is a flowchart of an example method for determining an SVR value using a PPG sensor;

FIG. 8 is a block diagram of an example system with multiple sensors for measuring SVR; and

FIG. 9 is a block diagram of an example environment for a health professional to measure SVR of a patient using a measurement device.

### DETAILED DESCRIPTION

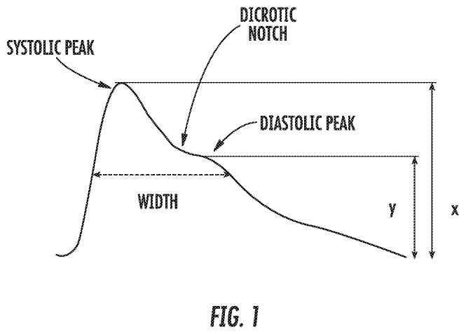

FIG. 1 is an example PPG waveform. Some example features are indicated on the waveform, including a systolic peak, a dicrotic notch, and a diastolic peak. The example PPG waveform can be recorded, for example, from a patient having a PPG sensor in contact with one of the patient's fingers.

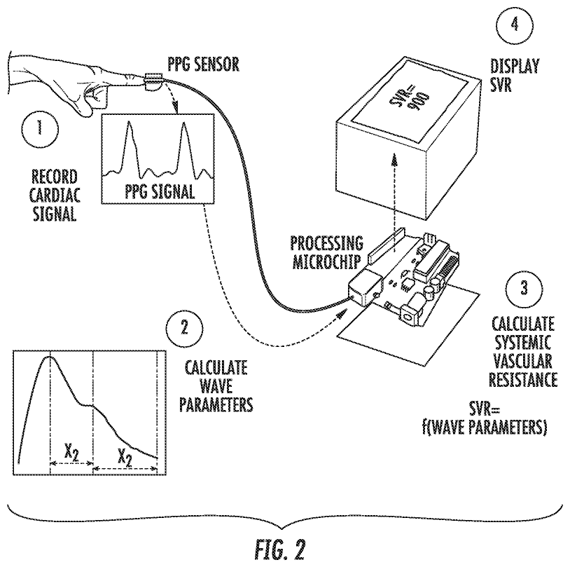

FIG. 2 is a block diagram illustrating the use of an example device for measuring SVR values. The device, which is referred to in some places of this document as an “EasySVR,” includes a PPG sensor, a processor (e.g., an Arduino processing chip), and an LCD screen. The sensor records the pulse wave (a cardiac signal) and then transmits it to the processing chip. The programmed chip calculates particular parameters from the wave (e.g. systolic peak-to-notch time), which then serve as inputs to a function programmed onto the same chip that outputs SVR. This output value is displayed on the LCD screen.

In order for the device to be an effective point-of-care device, the device can be configured to meet specifications for size, weight, power source, and time to give a result. First, the size of the device can be less than 6×6×6 in3, weigh less than three pounds, and be battery-operated so that EasySVR can be easily moved between patient rooms. EasySVR can have a battery life of at least one year on a commonly-used battery to make the device convenient for hospitals to use. In some examples, EasySVR has a detachable power cord and is configured to automatically charge the battery when plugged in. When the EasySVR is unplugged, the EasySVR runs on battery power.

Finally, EasySVR can be configured, by appropriate programming and hardware selection, to give a reading in less than five minutes to make it appropriate for use in emergency settings. Five minutes is an approximate time allotted in ED triage for measuring vital signs while the patient is seated, during which time noise from broad patient movements would be minimal. EasySVR can be used while vital signs are recorded, so it could be seamlessly incorporated into current triage procedure.

EasySVR can be configured, by virtue of appropriate selection of materials and hardware, to be affordable for screening in any appropriate medical setting, e.g. nursing homes, hospitals, hospital EDs, critical care and acute care areas, ambulances, and phyician's offices for various medical specialties. For example, the device may have a cost that is comparable to other professional point-of-care devices on the market. EasySVR can be configured, by virtue of appropriate programming, to report an accurate SVR value, e.g., an SVR value within 19% of a patient's true RHC value or as appropriate per government regulations.

Design Process

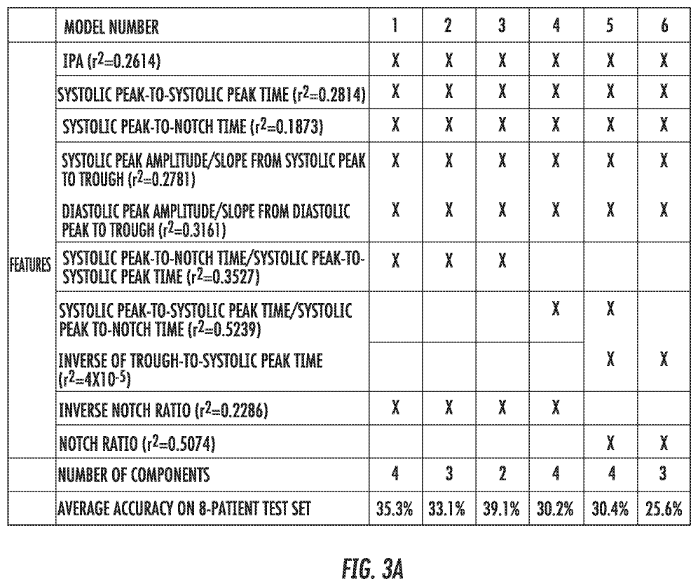

FIGS. 3A-C illustrate aspects of the process for developing a model for determining SVR values from cardiac waveforms and for implementing devices to use the model. The following section describes an example design process for designing software and hardware for an SVR measurement device. The devices, methods, and computer readable media described in this document can be designed and built according to any appropriate process. The following example design process is presented for purposes of illustration.

The point-of-care device comprises software and hardware components. We had two tasks in the software design process: to develop an algorithm that accurately predicts SVR and to program an Arduino microprocessor board to read in PPG data and output our algorithm-predicted SVR to an LCD screen. Pertaining to the hardware design, we needed to assemble the components required for processing in a compact device and facilitate user-friendliness.

In developing our algorithm, we first created a MATLAB script to analyze 26 clinic PPG tracings, which were scanned as PNG images. Our script required that we manually select the systolic peaks, dicrotic notches, and troughs (start of each wave) of the PPG tracings (see FIG. 1), which were then stored as fiducial points. We opted to manually detect key points to eliminate the possibility of erroneous automatic detection. The fiducial points were then used to calculate 34 various wave features, such as systolic peak-to-systolic peak time. We then plotted each feature versus SVR to determine which features were best correlated with SVR and should be utilized in our algorithm.

Using combinations of features, we trained our algorithm using data from 18 of our 26 patients. To divide patients into either the testing or training set, we ordered them by SVR and then assigned every third patient to be in the testing set. This allowed us to ensure that both sets had patients with a variety of SVR values and that our training set contained approximately twice as many samples as our testing set.

The resulting model uses Partial Least Squares Regression (PLSR). Because the size of our dataset is small and many of our features are collinear, we were at high risk of over-fitting models to our data. We therefore looked to regularization models that decrease the bias of our model to the 18 patients we trained on. PLSR works by projecting the predictor variables and the target variable, here SVR, onto latent subspaces that maximize the covariance between them.

The systolic peak, diastolic peak, and dicrotic notch are defined in FIG. 1, where “x” shows the systolic amplitude and “y” the diastolic amplitude. The start and end of this wave mark the start and end troughs, respectively. The systolic amplitudes and diastolic amplitudes are defined using the previous trough as a baseline, while the slopes are calculated using the next trough as a baseline, so the ratios do not reduce to a simple time feature but rather incorporate information about the locations of both the starting trough and ending trough for a wave. Notch ratio is the ratio of systolic peak-to-notch over notch-to-trough time. IPA, or inflection point area, is the ratio of A2 to A1, where A1 is the area under the wave from the start trough to the dicrotic notch and A2 is the area from the dicrotic notch to end trough.

We iterated through many possible algorithms by selecting various combinations of features and then performing PLSR with various numbers of components to generate possible algorithms. These possible algorithms were then tested on the 8 training set patients, and the error was recorded. The algorithms that gave less than 40% average error are shown in the table of FIG. 3A. As shown in the table, Model 6 with 7 features and 3 components yielded the lowest error on our test set, 25.6%, and we therefore used this algorithm in our final model.

In programming an Arduino board, we adapted existing code to find heart rate to incorporate the detection of more features and calculation of SVR using our algorithm. For our device, we created code to read in PPG data from the pulse sensor, analyze this data in real time to calculate pulse wave features, input these features into the algorithm, and then output the algorithm-predicted SVR to our LCD screen. To accomplish this, we wrote our code to collect 60 seconds' worth of data. The code analyzes the data using a 10-waveform sliding window and calculates the features in each window. Because this results in multiple values for each feature, the averaged feature value is input into our algorithm for each feature. We then wrote a script to output the algorithm-predicted SVR to the LCD screen.

Additionally, we wrote code to make the system more user-friendly. Specifically, scripts were written so that the various external components allowed the user to advance through different steps in using the device and communicated to the user what the device was doing. For example, when the device is turned on using the rocker switch, the LCD screen displays “Welcome to EasySVR,” letting the user know that the device is ready to be run.

In designing and constructing our physical device, we first needed to determine the necessary components and then appropriately connect them. These components include a standalone pulse sensor, an Arduino board, and an LCD screen. The pulse sensor and LCD screen were connected to pins on the Arduino board as specified by the code we used to program the Arduino board. We then added to this bare-bones model so that it was more user-friendly. FIG. 2 shows the connections in our final device.

Although some of the examples shown in this document show the EasySVR device with an LCD screen, in other examples, the EasySVR may have another type of display or may not have any display at all. For example, the EasySVR may include one or more PPG sensors, a computer system for determining SVR values, and an output port or communications system for outputting SVR values, e.g., so that the EasySVR can be integrated with or included within other systems that may already have a display.

To assemble the device, we designed a 5.12″×3.35″×1.97″ box in SolidWorks and 3D printed it. The interior of our box is divided into two compartments. The large main compartment contains the Arduino board, and all the wire connections between the Arduino board and the various external device components. Because we do not foresee the user having to open this compartment, we made it less accessible to reduce the chances of any wires becoming disconnected. The other, smaller compartment contains the 9V battery and is accessible by a removable battery cover, enabling a user to easily replace the battery when necessary. In some other examples, the box can have a single compartment or more than two compartments or any appropriate mechanical structure.

We also added a power switch, a push button, an LED indicator light, and a hook and loop finger strap to make our device user-friendly. The power switch will conserve battery power, and the push-button allows users to begin data collection when ready. An LED indicator light that blinks with the user's pulse allows the user to verify that the sensor is detecting their pulse wave. In some examples, the system includes a power cord, and the system can be configured to turn on when the power cord is plugged in and/or turn off when the power cord is unplugged.

Although the example system shown in FIG. 2 includes a hook and loop finger strap, in general, the EasySVR can include any appropriate mechanism for holding the PPG sensor in place while taking measurements. For example, the EasySVR may use an alligator clip (sized to receive a finger) or other type of mechanical fastener to hold the PPG sensor over the appropriate location on the finger. FIG. 3C shows three views of an example alligator clip. The first view 302 shows the alligator clip in a closed position, e.g., for storage or transport. The second view 304 shows the alligator clip in an open position, e.g., ready to receive a finger for taking measurements. The third view 306 shows the alligator clip closed on a finger, e.g., for taking measurements.

FIG. 3B shows the results of our testing for the specifications addressing the point-of-care goal. For the battery life specification, we found that the load current, determined by connecting an ammeter in series with the device while it was running, was 45.6 mA. EasySVR uses a single 9V battery, which has a capacity of 580mAh. We then determined battery life as 12.7 hours. Using the time to give a result of 75 seconds, we can estimate that EasySVR requires a battery change after 609 uses. Because we may not know how often a particular EasySVR device would be used in a hospital, we cannot conclusively say if EasySVR meets our original power-source specification. EasySVR's battery life is comparable to or better than many other clinical point-of-care devices.

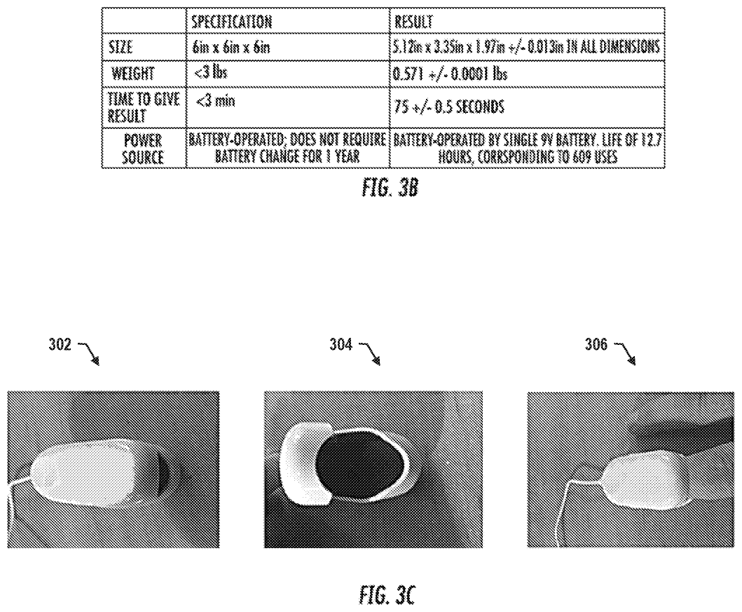

FIG. 4 shows an electrical schematic of an example implementation of the EasySVR device. By meeting our point-of-care specifications, which dealt with our device's portability and timeliness of SVR readings, we believe that our device can fit into the fast-paced emergency setting and other appropriate medical settings. Its small size, low weight, and battery-operated capability allow it to be quickly moved between rooms. Its rapid feedback in reporting the measured SVR value is also fitting for the emergency setting and other medical settings.

Obtaining more patient samples to train and test the algorithm is a future recommendation that would enable a reliable report of algorithm accuracy and likely lead to a more accurate algorithm than the one we were able to develop from just 26 patient sets. We believe increasing the number of samples used to train the algorithm will increase algorithm accuracy. An algorithm for which the percent error was less than 19%, 99% of the time would be appropriate for clinical use since it would approximate the accuracy obtainable by right-heart catheterization.

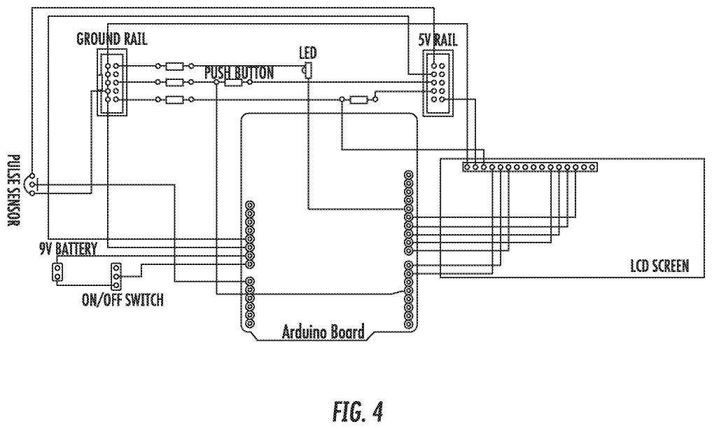

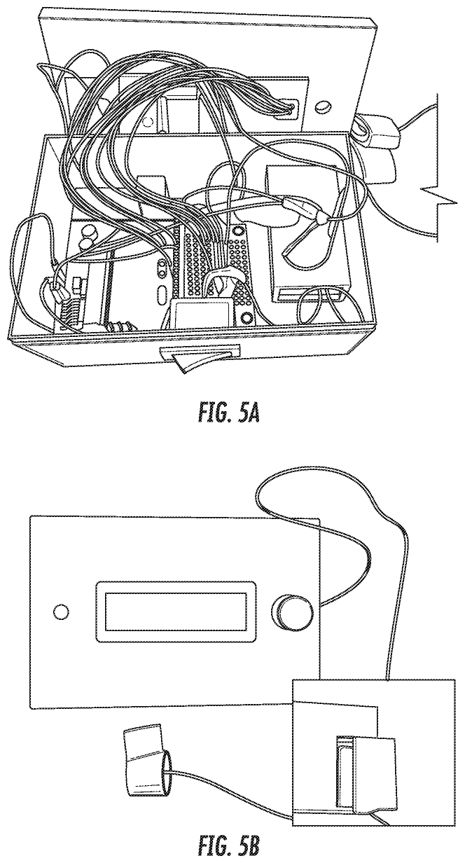

FIG. 5A depicts the inside of an example EasySVR, showing a microcontroller, battery leads, rocker switch, and protoboard with connections and rails. FIG. 5B depicts the outside of the example EasySVR, showing LCD screen, push button, LED, and pulse sensor. Inset picture shows bottom of box, where the user has access to replace the 9V battery.

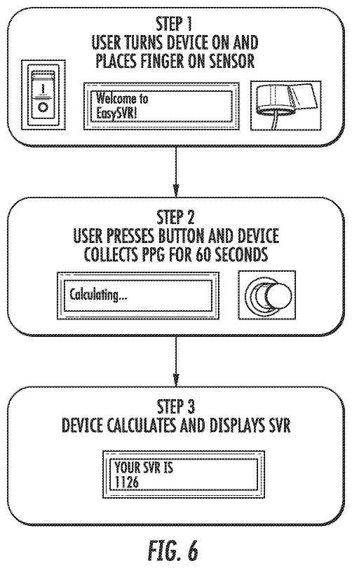

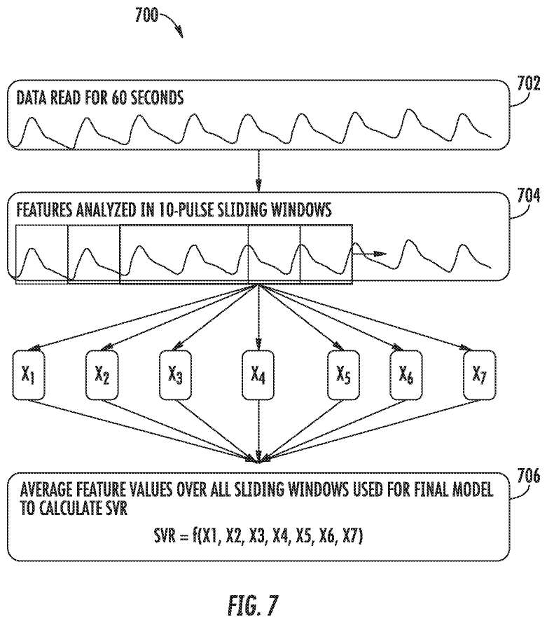

FIG. 6 is a flowchart showing the use of the device and user interface. The LCD screen displays “Welcome to EasySVR”, “Calculating . . . ”, and “Your SVR is: ####” during the process. FIG. 7 is a flowchart of an example method 700 for determining an SVR value using a PPG sensor. Method 700 includes taking PPG measurements over a certain amount of time, e.g., 60 seconds (702). Method 700 includes analyzing features from the PPG measurements in sliding windows over PPG pulses, e.g., windows of 10 pulses (704). Method 700 includes averaging feature values (e.g., a systolic peak amplitude, a diastolic peak amplitude, and a dicrotic notch amplitude) from the sliding windows and calculating an SVR using the averaged feature values and a model, e.g., a model determined as described above with reference to FIG. 3A (706).

The device can be used to diagnose a patient with sepsis or CHF. For example, the device can be configured to compare the SVR value to sepsis and CHF threshold values and diagnose the patient with sepsis if the SVR value is below the sepsis threshold and diagnose the patient with CHF if the SVR value is above the CHF threshold. In another example, the device can present the SVR value to a health professional for diagnosing the patient. The health professional can treat the patient for sepsis or CHF using any appropriate treatment, e.g., antibiotics and/or intravenous fluids for sepsis and lifestyle modification and/or medication for CHF. In some examples, the SVR value can be used in diagnosing other conditions, e.g., liver disease or kidney disease.

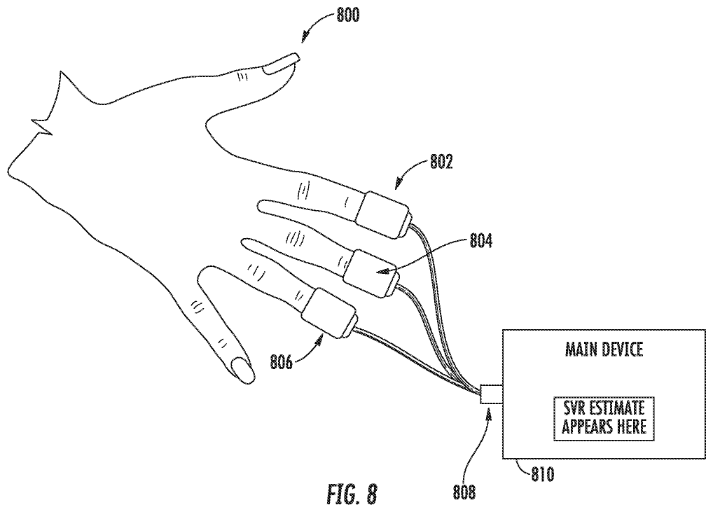

FIG. 8 is a block diagram of an example system 800 with multiple sensors for measuring SVR. System 800 includes a number of PPG sensors. Each PPG sensor can be embedded in a respective enclosure (e.g., an alligator clip or housing with a hook and a loop fastener) configured to fit finger sizes for respective fingers of a hand. The PPG sensors can be situated so that the sensors are in an appropriate location with respect to the fingers when the PPG sensors are secured to the fingers of the hand. For example, each sensor can include an optical emitter/sensor pair that faces the pad of a respective finger when the PPG sensors are secured to the fingers of the hand.

System 800 includes PPG sensors 802, 804, and 806 for the ring finger, the middle finger, and the index finger. PPG sensors 802, 804, and 806 are connected to a port 808, e.g., a universal serial bus (USB) port, which is connected to a data processing unit 810. In some examples, each PPG sensor sends independent data streams. Data processing unit 810 executes an algorithm that determines an SVR value for each data stream. Data processing unit 810 then determines an SVR value for a patient based on the SVR values, e.g., by averaging the SVR values.

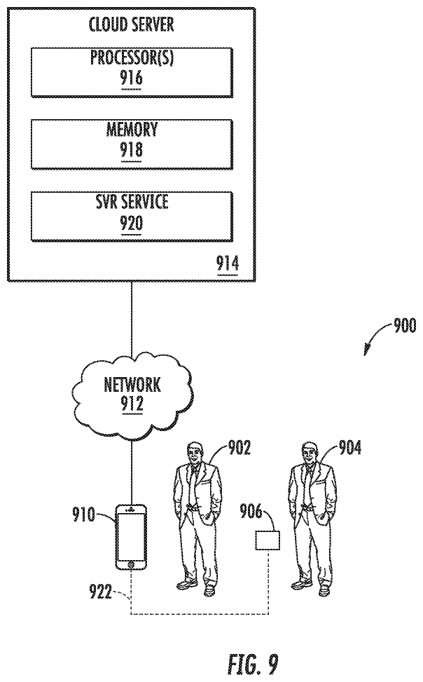

FIG. 9 is a block diagram of an example environment 900 for a health professional 902 to measure SVR of a patient 904 using a measurement device 906. Device 906 has a PPG sensor, e.g., a light emitting diode (LED) and an optical sensor matched to the LED. The LED can be configured to emit light of any appropriate wavelength, e.g., in the range of about 805 nm to 905 nm (infrared), or of about 495 nm to 570 nm (green), or of about 620 nm to 750 nm (red). In some examples, device 906 includes one or more LEDs, e.g., a red LED and an infrared LED which are both wired and oriented to emit light on a same target spot at the same time.

Device 906 can include other optional features, e.g., a battery, a strap or other mechanical feature to secure device 906 to a finger, and a removable memory card such as a Secure Device (SD) card for storing data from the PPG sensor. In some examples, device 906 includes a light-blocking box or cloth or other structure to block or dim ambient light from reaching the PPG sensor. For example, device 906 may include a shielded cable. Typically, device 906 and the PPG sensor and an optional sensor cable will be protected from stray light (e.g., fluorescent light) and other traditional hospital interferences or other types of interferences.

Health professional 902 checks that device 906 is powered (e.g., battery is charged) and cleaned and then places or assists patient 904 in placing one of patient 904's fingers in an appropriate location of device 906. Health professional 902 initiates an SVR measurement, e.g., by pressing a power button or a start button. Device 906 begins taking measurements using the PPG sensor.

In some examples, device 906 includes at least one processor and a display. The processor can be programmed to determine an SVR value based on the measurements from the PPG sensor, e.g., as described above with reference to FIG. 7. The processor can be programmed to display a result on the display, e.g., the SVR value or other appropriate indication based on the SVR value.

In some examples, device 906 includes a communications system for transmitting the PPG measurements to another device 910 over a wired or wireless communications link 922. Device 910 can be, e.g., a tablet computer, laptop computer, or other appropriate user device having a display. Health professional 902 can use device 910 to receive PPG measurements from device 906.

Device 910 can be programmed to determine an SVR value based on the measurements from the PPG sensor. Alternatively, device 910 or device 906 can be programmed to transmit the measurements over a data communications network 912 (e.g., the Internet) to a cloud server 914. Cloud server 914 comprises at least one processor 916 and memory 918 and is configured to implement an SVR service 920. SVR service 920 receives PPG measurements and determines SVR values and can send SVR values back to device 910, e.g., so that device 910 can display the SVR values or other appropriate indicators based on the SVR values (e.g., a color, symbol or a numeric value corresponding to a range containing the SVR value,). Each of SVR service 920, device 910, and device 906 can be configured to protect stored patient information, e.g., in accordance with appropriate regulations such as the Health Insurance Portability and Accountability Act (HIPAA).

Conclusion

This document describes a point-of-care, affordable, and clinically applicable device, EasySVR, to measure a patient's SVR non-invasively. To meet this goal, we chose to use the non-invasive finger photoplethysmograph. The intended application of EasySVR is for assessing if a patient's SVR is abnormally low or high during triage in the emergency department or patient examination in other appropriate medical setting, as these are indicators of sepsis and heart failure, respectively, and other acute and chronic illnesses such as kidney disease and liver disease. Our specifications ensure that EasySVR is practical and appropriate for this application. Meeting our specifications for size, weight, time to give a result, and power source ensure that EasySVR is a point-of-care device that is easily transported and quick to report results. We also met the specification setting an upper limit of device cost, which can be useful to ensure affordability of the device in hospitals and other appropriate medical settings.

Appropriate accuracy can be achieved by following a few recommendations for future work. First, the accuracy of the algorithm could be improved by using more patient data to develop the algorithm. We suggest training on a sufficient number of samples requiredto achieve an appropriate level of accuracy for particular implementations, for example, training on 152 samples and testing on 76 samples in order to improve the algorithm accuracy and report an accuracy value at 80% statistical power as discussed previously. Second, we suggest either using a clinical PPG sensor in EasySVR instead of a commercial Adafruit pulse sensor, or training the algorithm on PPG tracings collected directly from the Adafruit sensor. By following these recommendations, we believe EasySVR can be improved such that it is appropriate for clinical use and would be a potentially life-saving screening tool in emergency departments.

Accordingly, while the methods, systems, and computer readable media have been described herein in reference to specific embodiments, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein.

Various combinations and sub-combinations of the structures and features described herein are contemplated and will be apparent to a skilled person having knowledge of this disclosure. Any of the various features and elements as disclosed herein may be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

It is understood that various details of the presently disclosed subject matter may be changed without departing from the scope of the presently disclosed subject matter. Furthermore, the foregoing description is for the purpose of illustration only, and not for the purpose of limitation.

The disclosure of each of the following references is incorporated herein by reference in its entirety.

### REFERENCES

[1] Lee, Q. Y., Chan, G. S., Redmond, S. J., Middleton, P. M., Steel, E., Malouf, P., . . . Lovell, N. H. (2011). Multivariate classification of systemic vascular resistance using photoplethysmography. Physiological Measurement, 32, 1117-1132. doi: 10.1088/0967-3334/32/8/008
[2] Mayo Clinic Staff. (July 2014). Sepsis. Retrieved from: http://www.mayoclinic.org/diseasesconditions/sepsis/basics/sym ptoms/con-20031900
[3] Filbin, M. R., Arias, S. A., Camargo, C. A., Barche, A., & Pallin, D. J. (March 2014). Sepsis visits and antibiotic utilization in the U.S. emergency departments. Critical Care Medicine, 42(3), 528-535. doi: 10.1097/CCM.0000000000000037
[4] U.S. Department of Health and Human Services. (3 Dec. 2013). Heart failure fact sheet. Centers for Disease Control and Prevention. Retrieved from: http://www.cdc.gov/dhdsp/data_statistics/fact_sheets/fs_heart_failure.htm
[5] Longworth, D, & Stoller, J. K. (2003). Nausea, vomiting, and confusion in a 74-year-old woman. In The Cleveland Clinic Internal Medicine Case Reviews (Section VII: Nephrology). Retrieved from: Google Books
[6] Cleveland Clinic. (2011). B-type natriuretic peptide (BNP) blood test. Retrieved from: http://my.clevelandclinic.org/services/heart/diagnostics-testing/laboratory-tests/b-typenatriuretic-peptide-bnp-bloodtest
[7] Collins, S. P., Phillip, L. D., Pang, P. S., & Gheorghiade, M. (June 2013). The role of the emergency department in acute heart failure clinical trials-enriching patient identification and enrollment. American Heart Journal, 165(6), 902-909. doi: 10.1016/j.ahj.2013.03.009
[8] Cotton, P. (6 Jul. 1994). Studies question right heart catheterization. The Journal of the American Medical Association, 272(1), 8. doi: 10.1001/jama.1994.03520010016004.
[9] Elgendi, M. (2012). On the analysis of fingertip photoplethysmography. Current Cardiology Reviews, 8, 14-25.
[10] Awad, A. A., Haddadin, A. S., Tantawy, H., Badr, T. M., Stout, R. G., Silverman, D. G., & Shelley, K. H. (2007). The relationship between photoplethysmographic waveform and systemic vascular resistance. Journal of Clinical Monitoring and Computing, 21(6), 365-372. doi:10.1007/s10877-007-9097-5
[11] Finometer™ User's Guide. (2002). Retrieved from: http://nuigalway.ie/psy/documents/finometer_manual.pdf
[12] Asrar ul Haq, M., Hare, D. L., Wong, C., Hayat, U., & Barlis, P. (2014). Treatment of hypertension in heart failure with preserved ejection fraction. Current Hypertension Reviews, 10, 142-148. doi:10.2174/1573402111666141217112216
[13] Finapres Finometer PRO. Sale Medical. Retrieved from: http://www.salemedical.com/products/FINAPRES-FINOMETER-PRO.html
[14] Medical Solutions. GO2 9570 Pulse Oximeter Fingertip. Retrieved from: https://www.4m dm edical.com/index. php/catalog/product/view/id/136733/s/noningo2-9570-pulse-oximeter-fingertip/
[15] Ramirez, M. F. L., Tibayan, R. T., Marinas, C. E., Yamamoto, M. E., & Caguioa, E. V. S. (2005). Prognostic value of hemodynamic findings from impedance cardiography in hypertensive stroke. American Journal of Hypertension, 18, 65S-72S. doi: 10.1016/j.amjhyper.2004.11.027
[16] Dobbin, K. K., Simon, R. M. (2011). Optimally splitting cases for training and testing high dimensional classifiers. BMC Medical Genomics, 4:31 doi:10.1186/1755-8794-4-31.
[17] Gowen, A. A., Downey, G., Esquerre, C., O'Donnell, C. P. (2010). Preventing over-fitting in PLS calibration of near-infrared (NIR) spectroscopy data using regression coefficients. Journal of Chemometrics, 25, 375-381 doi: 10.1002/cem.1349
[18] Stratasys. (2013). uPrint SE. Retrieved from: http://www.stratasys.com/-/media/Main/Secure/System_Spec_Sheets-SS/DimensionProductSpecs/uPrintSESellSheet-INTL-ENG-10-13V020WEB.pdf
[19] Duracell MN1604 9 Volt Coppertop Battery. BatteryStore. Retrieved from: http://www.batterystore.com/duracell-mn1604-9-volt-coppertop-battery/
[20] NovaBiomedical. StatStrip® Connectivity Point-of-Care Glucose Analyzers. Retrieved from: http://www.novabiomedical.com/products/statstrip-1-75-connectivity-point-ofcare-glucose-analyzers/[21]
[21] ATT Labs. Dimension 1200es Material Cartridges. Retrieved from: http://www.aetlabs.com/product/dimension-1200es-sst-model-material/[22]
[22] HyLown Consulting. (2015). Test 1 Proportion: 1-Sample, 1-Sided. Retrieved from: http://powerandsamplesize.com/Calculators/Test-1-Proportion/1-Sample-1-Sided

1. A device for measuring systemic vascular resistance (SVR), the device comprising:
a photoplethysmography (PPG) sensor;
a display device; and
a computer system programmed to perform operations comprising:
determining a plurality of wave parameters from a cardiac waveform signal detected by the PPG sensor, wherein the wave parameters include at least a systolic peak amplitude, a diastolic peak amplitude, and a dicrotic notch amplitude;
determining an SVR value based on the wave parameters; and
displaying the SVR value on the display device.

2. The device of claim 1, wherein the wave parameters include at least:
inflection point area (IPA);
systolic peak-to-systolic peak time;
systolic peak-to-notch time;
systolic peak amplitude/slope from systolic peak to trough;
diastolic peak amplitude/slope from diastolic peak to trough;
inverse of trough-to-systolic peak time; and
notch ratio.

3. The device of claim 1, wherein determining the plurality of wave parameters comprises determining the plurality of wave parameters for each window of a plurality of sliding windows over the cardiac waveform signal.

4. The device of claim 3, wherein determining the SVR value comprises determining average wave parameters for each of the wave parameters based on the wave parameters for each window of the sliding windows and determining the SVR value based on the average wave parameters.

5. (canceled)

6. (canceled)

7. (canceled)

8. The device of claim 1, comprising an LED indicator light, wherein the computer system is programmed to cause the LED indicator light to blink with a pulse detected from the cardiac waveform signal.

9. (canceled)

10. The device of claim 1, comprising a plurality of finger PPG sensors, wherein the computer system is programmed to determine a finger SVR value for each of the finger PPG sensors and display an average of the finger SVR values on the display device.

11. A method for measuring systemic vascular resistance (SVR), the method comprising:
determining, by a computer system coupled to a photoplethysmography (PPG) sensor and a display device, a plurality of wave parameters from a cardiac waveform signal detected by the PPG sensor, wherein the wave parameters include at least a systolic peak amplitude, a diastolic peak amplitude, and a dicrotic notch amplitude;
determining, by the computer system, an SVR value based on the wave parameters; and
displaying the SVR value on the display device.

12. The method of claim 11, wherein the wave parameters include at least:
inflection point area (IPA);
systolic peak-to-systolic peak time;
systolic peak-to-notch time;
systolic peak amplitude/slope from systolic peak to trough;
diastolic peak amplitude/slope from diastolic peak to trough;
inverse of trough-to-systolic peak time; and
notch ratio.

13. The method of claim 11, wherein determining the plurality of wave parameters comprises determining the plurality of wave parameters for each window of a plurality of sliding windows over the cardiac waveform signal.

14. The method of claim 13, wherein determining the SVR value comprises determining average wave parameters for each of the wave parameters based on the wave parameters for each window of the sliding windows and determining the SVR value based on the average wave parameters.

15. The method of claim 11, wherein the computer system is housed in a device comprising a battery and a housing divided into at least first and second compartments, wherein the computer system is stored in the first compartment, and wherein the battery is stored in the second compartment and the second compartment is accessible by a removable battery cover.

16. (canceled)

17. (canceled)

18. The method of claim 11, comprising causing an LED indicator light to blink with a pulse detected from the cardiac waveform signal.

19. (canceled)

20. The method of claim 11, comprising determining a finger SVR value for each finger PPG sensor of a plurality of PPG sensors and displaying an average of the finger SVR values on the display device.

21. One or more non-transitory computer readable mediums storing instructions for a device comprising at least one processor that, when executed by the at least one processor, cause the at least one processor to perform operations comprising:
determining a plurality of wave parameters from a cardiac waveform signal detected by a photoplethysmography (PPG) sensor, wherein the wave parameters include at least a systolic peak amplitude, a diastolic peak amplitude, and a dicrotic notch amplitude;
determining an SVR value based on the wave parameters; and
displaying the SVR value on a display device.

22. The method of claim 11 comprising:
diagnosing a patient with congestive heart failure (CHF), sepsis, kidney disease, or liver disease based on the SVR value.

23. The method of claim 22, wherein diagnosing the patient comprises comparing, by the computer system, the SVR value to a sepsis threshold value and diagnosing the patient with sepsis if the SVR value is below the sepsis threshold value.

24. The method of claim 22, wherein diagnosing the patient comprises comparing, by the computer system, the SVR value to a CHF threshold value and diagnosing the patient with CHF if the SVR value exceeds the CHF threshold value.

25. The method of claim 22, wherein diagnosing the patient comprises presenting the SVR value on a display device to a health professional for diagnosing the patient.

26. The method of claim 22, comprising treating the patient for CHF, sepsis, kidney disease, or liver disease based on diagnosing the patient with CHF, sepsis, kidney disease, or liver disease.

27. A system comprising a device according to claim 1 and further comprising:
a communications system for transmitting a plurality of measurements from the PPG sensor.

28. (canceled)

29. (canceled)

30. (canceled)
